(19) United States
(12) Patent Application Publication
(10) Pub. No.: US 2006/0241741 A1
(43) Pub. Date: Oct. 26, 2006

(54) connecting system for connecting a stent to a radiopaque marker and a process for the production of a connection between a stent and two or more radiopaque markers

(76) Inventor: Daniel Lootz, rostock (DE)

Correspondence Address: HAHN LOESER & PARKS, LLP, One GOJO Plaza, Suite 300, AKRON, OH, 44311-1076, US

(21) Appl. No.: 10/559,592

(22) PCT Filed: Jun. 1, 2004
(86) PCT No.: PCT/EP04/05883

§ 371 Date: Jul. 6, 2006

(30) Foreign Application Priority Data
Jun. 2, 2003 (DE) 10325678.4

(51) Int. Cl. A61F 2/90

(52) U.S. Cl. 623/1.34

(57) ABSTRACT

The invention concerns a connecting system for connecting a stent to a radiopaque marker. The intention is to provide a connecting system which allows the stent to be connected to a radiopaque marker without a worsening of the mechanical properties of the stent and which at the lowest possible level of structural complication and expenditure provides a holding force which is adequate for probing with and implantation of the stent. That is achieved in that the connecting system includes at least one gripping connection comprising a gripping element and a clamping element.

### BACKGROUND OF THE INVENTION

The invention concerns a connecting system for connecting a stent to a radiopaque marker and an associated process for the production of a connection between a stent and two or more radiopaque markers.

Stents, in particular for coronary use, are usually formed from metals or metal alloys, for example stainless 316L steel or nitinol but also special polymer materials. It will be noted however that the materials used suffer from the disadvantage that they cannot be radiologically detected at all, or only with difficulty. However, the X-ray process represents by far the most powerful instrument for monitoring the implantation procedure, the relative position and the expansion condition of the stent.

To provide a remedy, it is known inter alia, for the stent to be coated with radiopaque materials. They include in particular metals such as platinum, palladium, silver, lanthanides and alloys thereof. It has been found however that an indifferent coating over parts or indeed the entire surface of the stent has an adverse effect on the mechanical properties of the basic structure of the stent. Particularly when using self-expanding stents, expansion in the desired fashion is impeded and can at most be compensated by additional structural measures on the stent design.

It is known from U.S. Pat. No. 6,022,374 for the radiopaque marker not to be mounted directly on the basic structure but on a region which is isolated therefrom and which is not an impediment in terms of stent expansion. Provided for that purpose is a frame element which is fitted on to that specific portion of the basic structure and includes a circular opening with inwardly directed points. The marker is inserted into that opening.

An aspect of the present invention is to provide a connecting system which allows the stent to be connected to a radiopaque marker without worsening the mechanical properties of the stent and which at the lowest possible level of structural complication and expenditure, affords a holding force which is adequate for probing with and implantation of the stent.

### BRIEF SUMMARY OF THE INVENTION

The stent according to the invention provides a connecting system for connecting a stent to a radiopaque marker that includes at least one gripping connection comprising a gripping element and a clamping element. It has surprisingly been found that the structural measures, which are simple in themselves, for implementing the connecting system according to the invention, both provide a holding force for the marker, which is adequate at least during probing with and implantation of the stent, and also do not have any or have only negligibly slight influences on the mechanical properties of the stent.

The connecting system according to the invention has proven to be particularly advantageous in terms of use of self-expanding stents. It is precisely here that the structural measures for bonding a marker in place must have as little influence as possible or no influence at all on the mechanics of the basic structure. In that respect, care must be taken to ensure in particular that, when establishing a connecting system, the lowest possible level of thermal and/or mechanical influences is exerted on the basic structure as otherwise the property of the material forming the basic structure, to be able to act as a shape memory material, can be undesirably influenced or totally lost. That can be ensured by means of the connecting system according to the invention.

The connecting system according to the invention can also be used in relation to biodegradable basic structures of the stent, for example consisting of magnesium alloys. In that respect, the position and shape of the connecting system has no influence or only a slight influence on the degradation behaviour of the basic structure so that uniform decomposition takes place in the living organism.

It is further preferred that the marker itself is in the form of a gripping or clamping element of the gripping connection. There is accordingly, no need for gripping or clamping elements of a different material to be formed on the marker by previous working steps.

In a further configuration of the concept of the invention, the marker is formed from a biocompatible material if the marker is designed to remain in the body of the patient for a prolonged period of time or permanently. It is precisely in the case of biodegradable stents that medium-term and long-term complications as a consequence of rejection reactions on the part of the body are to be obviated in that way. It is particularly preferred if the marker entirely or in parts comprises one or more metals from the group Ta, Nb, Zr, Hf, Mo, W, Au, Pt, Ir, rare earths or alloys thereof, in particular PtIr. The specified materials are distinguished by good availability, a high level of biocompatibility and easy workability.

It is further preferred if the gripping or clamping element of the gripping connection is formed on the basic structure of the stent, that is to say, it is not a constituent part thereof. In that way it is possible to minimize or exclude highly effectively troublesome influences of the connecting system on the transition from the non-expanded condition of the stent into the expanded condition, and also on the mechanical stability of the stent in the non-expanded condition and the expanded condition.

It is further preferred if the gripping or clamping element is arranged at both (proximal) ends of the stent. That can simplify the production process as the proximal ends of the stent are more easily accessible. It is also advantageous for the connecting system to be integrated into the basic structure in such a way that it does not project or projects at most to a slight extent in a radial direction beyond the dimensions of the peripheral wall of the basic structure. In that way, by virtue of the specific position, it is possible to ensure that the gripping connection does not project out of the plane of the basic structure and therefore could not result in vessel damage in the probing or implantation procedure.

A further aspect of the invention concerns a process for the production of a connection between the stent and two or more radiopaque markers, more specifically using a connecting system having the above-mentioned features. The process is distinguished in that:

(a) two or more markers are connected together by way of a positioning element so that the markers are aligned with their gripping or clamping elements with the corresponding gripping or clamping elements of the stent,

(b) in a working step the markers are placed with their gripping or clamping elements on to the corresponding gripping or clamping elements of the stent, and

(c) then the connection between the positioning element and the gripping or clamping elements of the marker is separated.

The above-outlined process implementation means that a plurality of markers can be simultaneously or almost simultaneously connected to a stent in a short time. The particular design configuration by means of a positioning element connecting the individual markers means that it is possible to achieve a high level of positioning accuracy, that is to say the markers are sufficiently precisely centered and fixed for radiological examination methods.

The term ‘gripping element’ is used to denote an open frame structure whose short-term deflection out of a rest position leads to the production of a return force (gripping force).

The term ‘clamping element’ is used to denote a structural element which is adapted in its shaping and dimensions to being received in the gripping element.

The term ‘gripping connection’ is used to denote a connection comprising a gripping element and a clamping element in which the clamping element, after being received in the gripping element, is held in force-locking relationship in contact with the gripping element.

The term ‘basic structure’ is used to denote all structural components of the stent which are exposed to mechanical loadings in the transition from the non-expanded condition of the stent into the expanded condition and which contribute to the mechanical stability of the stent in the non-expanded and expanded conditions.

### BRIEF DESCRIPTION OF THE SEVERAL VIEWS OF THE DRAWINGS

The invention is described in greater detail hereinafter by means of embodiments by way of example and with reference to the accompanying drawings in which:

FIG. 1 shows a proximal end portion of a stent with a connecting system according to the invention,

FIGS. 2a-c show an enlarged section from the stent of FIG. 1 in the region of the connecting system according to the invention prior to and after receiving two markers of different dimensions,

FIGS. 3a-c show three alternative embodiments by way of example of gripping elements, and

FIG. 4 is a view in principle to explain the process according to the invention for the production of a connection between a stent and two or more radiopaque markers.

### DETAILED DESCRIPTION OF THE INVENTION

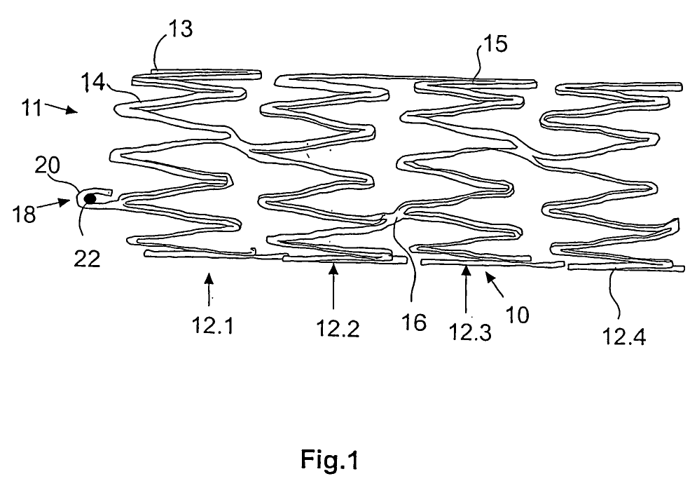

FIG. 1 diagrammatically shows a portion of a stent 10 in the region of its proximal end 11 and more specifically as a plan view on to a development of its peripheral wall 13 which extends in a tubular configuration. A basic structure 15 of the stent 10 is composed of a plurality of segments 12.1, 12.2, 12.3, 12.4 which in turn comprise legs 14 extending in a meander configuration in the peripheral direction, the individual segments 12.1, 12.2, 12.3, 12.4 being connected together in selected regions by way of bridges 16. The illustrated stent design is self-evidently only to be viewed by way of example. It will be appreciated that any variations—as are known in a large number of different forms from the state of the art—can be used in conjunction with the connecting system according to the invention.

The connecting system 18 includes a gripping element 20 and a corresponding clamping element 22. The gripping element 20 is arranged at a terminal position at the proximal end 11 of the stent 10. In the specific case the clamping element is formed from a radiopaque material and thus in itself represents the radiopaque marker.

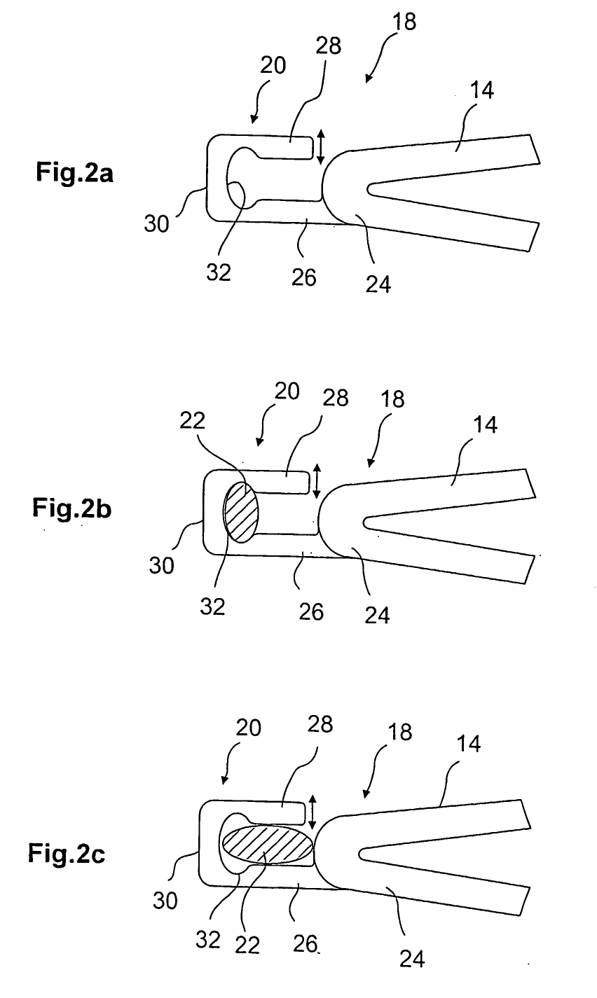

Further details can be seen from FIGS. 2a, 2b and 2c showing an enlarged section from the stent 10 of FIG. 1 in the region of the connecting system 18. The gripping element 20 is formed in a terminal curvature region 24 of a leg 14 on the basic structure 15 of the stent 10. It is possible to have recourse to conventional connecting procedures for the operation of shaping the gripping element on the stent, and those procedures are to be respectively individually matched to the selected materials of the stent 10 and the clamping element 20. A choice of material for the gripping element 20 is limited only by the following premises:

the material must be biocompatible, by virtue of its use, and

the material must be suitable for the implementation of an open frame structure whose brief deflection of a frame element out of the rest position results in the production of a return force (gripping force) without the structure breaking.

It is also possible for the gripping element 20 to be formed from a biodegradable material, in particular if the stent 10 is also to have those properties.

As can be seen from FIGS. 2a-2c the gripping element 20 is in the form of a clip-shaped element of which one arm 26 is formed on the stent 10 directly in the curvature region 24 thereof. A second, somewhat shorter arm 28 admittedly faces in the direction of the curvature region 24, but is not joined thereto. An extent of the gripping element 20, which is radial with respect to a longitudinal axis of the stent 10, corresponds at maximum to the corresponding dimensions of the basic structure 15 of the stent 10, that is to say the gripping element 20 does not project beyond the individual legs 14 of the peripheral wall 13. That makes it possible to minimize the danger of any vessel injuries.

The clip-shaped gripping element 20 has in its end 30 an inner circular enlargement 32 which serves as a receiving means for the clamping element 22—here the radiopaque marker. The region 32 can be adapted in shape to the clamping element 22 so that the clamping element 22 is held in positively locking and force-locking relationship. FIGS. 2b and 2c show two clamping elements 22 of different kinds of dimensions which can be received by the gripping element 20. As indicated by the arrow in FIG. 2a, the second arm 28 exhibits an elastic behaviour when it is deflected out of its rest position. Thus, if the second arm 28 is moved away from the first arm 26 prior to or during placement of the clamping element 22, it moves back into its original position again however by virtue of its elastic properties, with a given return force. That return force can be used at least in part to hold the clamping element 22 after it has been received in the enlargement 32.

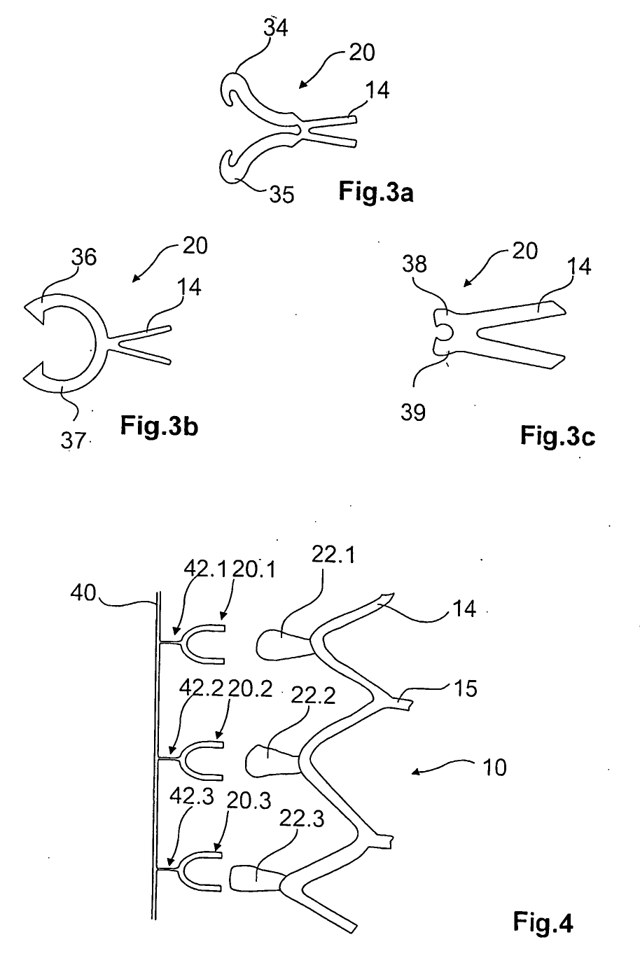

FIGS. 3a through 3c show further alternative embodiments of the gripping element 20. FIG. 3a shows a gripping element 20 having two hook-shaped arms 34, 35 defining a funnel-shaped opening. This embodiment of the gripping element 20 is suitable for clamping elements involving a leaf-shaped or band-shaped contour. FIG. 3b discloses a gripping element 20 having two arms 36, 37 which provide a C-shaped opening for a clamping element. The clamping element for such a gripping element 20 is preferably cylindrical or semicylindrical. Finally, FIG. 3c shows a gripping element 20 having two short arms 38, 39 defining a u-shaped opening. This embodiment is suitable in particular for narrow-cylindrical clamping elements such as small wire portions and the like.

The markers used comprise a biocompatible material. They can comprise entirely or in parts one or more of the metals from the group Ta, Nb, Zr, Hf, Mo, W, Au, Pt, Ir, rare earths or alloys thereof, for example PtIr. They are of a shape which is distinguishable in at least one axial direction in space, that is to say they do not involve in particular spherical symmetry, for better radiological distinguishability.

In the above-depicted embodiments the clamping element 22 was in each case the radiopaque marker itself.

FIG. 4 shows an embodiment in which the radiopaque marker is in the form of a gripping element 20.1, 20.2, 20.3. In this case a plurality of drop-shaped clamping elements 22 are formed on the basic structure 15 of the stent 10. For this specific case, it has been found to be particularly useful if the radiopaque marker comprises tantalum as that material has high X-ray density and is easily deformable and also enjoys adequate biocompatibility. In precisely the same fashion it would be possible to use Nb, Zr, Hf, Mo, W, Au, Pt, Ir, rare earths or alloys thereof.

A connection between the stent 10 and the radiopaque marker can be effected, for example, in such a way that a respective clamping element is placed in a gripping element by means of a positioning system, from a stocking reservoir containing a large number of clamping elements. Positioning systems of that kind are known in principle from the state of the art and can be designed in a highly variable fashion so that there is no need to discuss them in greater detail at this juncture.

If two or more radiopaque markers are to be connected to a stent at the same time, it is then possible to proceed as diagrammatically indicated in FIG. 4. The total of three gripping elements 20.1, 20.2, 20.3 of a radiopaque material are placed by means of a positioning element 14 simultaneously or almost simultaneously on the total of three clamping elements 22 which are formed on the basic structure 11 of the stent 10. The positioning element 14 is connected to each of the gripping elements 20.1, 20.2, 20.3 by way of a small respective limb 42.1, 42.2, 42.3. After the gripping elements 20.1, 20.2, 20.3 have been fitted on to the respectively corresponding clamping elements 22.1, 22.2, 22.3 the limbs 42.1, 42.2, 42.3 are separated, for example by means of laser cutting.

We claim:

1. A connecting system for connecting a stent to a radiopaque marker, the connecting system comprising at least one gripping connection comprising a gripping element and a clamping element.

2. A connecting system as set forth in claim 1, wherein the radiopaque marker is in the form of a gripping or clamping element of the gripping connection.

3. A connecting system as set forth in claim 1, wherein the marker is formed from a biocompatible material.

4. A connecting system as set forth in claim 3, wherein the marker at least in part comprises one or more metals selected from the group consisting of Ta, Nb, Zr, Hf, Mo, W, Au, Pt, Ir, rare earths and alloys thereof.

5. A connecting system as set forth in claim 4, wherein the marker at least in part comprises PtIr.

6. A connecting system as set forth in claim 1, wherein the gripping or clamping element of the gripping connection is formed on a basic structure of the stent.

7. A connecting system as set forth in claim 6, wherein the connecting system is integrated into the basic structure in such a way that it does not project or projects at most to a slight extent in a radial direction beyond the dimensions of a peripheral wall of the basic structure.

8. A connecting system as set forth in claim 6, wherein the gripping or clamping element is arranged at the proximal end of the stent.

9. A connecting system as set forth in claim 6, wherein the gripping or clamping element is formed from a biodegradable material.

10. A connecting system as set forth in claim 1, wherein the stent is self-expanding.

11. A connecting system as set forth in claim 1, wherein the stent is biodegradable.

12. A connecting system as set forth in claim 11, wherein the stent is formed entirely or in parts from a biodegradable Mg-alloy.

13. A process for the production of a connection between a stent and two or more radiopaque markers by means of a connecting system as set forth in claim 1, wherein:
(a) two or more markers are connected together by way of a positioning element so that the markers are aligned with their gripping or clamping elements with the corresponding gripping or clamping elements of the stent,
(b) in a working step the markers are placed with their gripping or clamping elements on to the corresponding gripping or clamping elements of the stent, and
(c) then the connection between the positioning element and the gripping or clamping elements of the marker is separated.

14. A process for the production of a connection between a stent and two or more radiopaque markers by means of a connecting system as set forth in claim 4, wherein:
(a) two or more markers are connected together by way of a positioning element so that the markers are aligned with their gripping or clamping elements with the corresponding gripping or clamping elements of the stent,
(b) in a working step the markers are placed with their gripping or clamping elements on to the corresponding gripping or clamping elements of the stent, and
(c) then the connection between the positioning element and the gripping or clamping elements of the marker is separated.

15. A process for the production of a connection between a stent and two or more radiopaque markers by means of a connecting system as set forth in claim 9, wherein:
(a) two or more markers are connected together by way of a positioning element so that the markers are aligned with their gripping or clamping elements with the corresponding gripping or clamping elements of the stent,
(b) in a working step the markers are placed with their gripping or clamping elements on to the corresponding gripping or clamping elements of the stent, and
(c) then the connection between the positioning element and the gripping or clamping elements of the marker is separated.

16. A process for the production of a connection between a stent and two or more radiopaque markers by means of a connecting system as set forth in claim 10, wherein:
(a) two or more markers are connected together by way of a positioning element so that the markers are aligned with their gripping or clamping elements with the corresponding gripping or clamping elements of the stent,
(b) in a working step the markers are placed with their gripping or clamping elements on to the corresponding gripping or clamping elements of the stent, and
(c) then the connection between the positioning element and the gripping or clamping elements of the marker is separated.

17. A connecting system as set forth in claim 2, wherein the marker is formed from a biocompatible material.

18. A connecting system as set forth in claim 17, wherein the marker at least in part comprises PtIr.

19. A connecting system as set forth in claim 9, wherein the stent is self-expanding.

20. A connecting system as set forth in claim 19, wherein the stent is biodegradable.
